(19) United States
(12) Patent Application Publication
(10) Pub. No.: US 2012/0109060 A1
(43) Pub. Date: May 3, 2012

(54) LAMINAR VALVE FLOW MODULE

(75) Inventors: George Kick, Casa Grande, AZ (US); Sean Cline, Arizona City, AZ (US)

(73) Assignee: EP Dynamics, Inc., Los Angeles, CA (US)

(21) Appl. No.: 13/098,460

(22) Filed: Apr. 30, 2011

Related U.S. Application Data
(63) Continuation-in-part of application No. 12/779,951, filed on May 13, 2010.
(60) Provisional application No. 61/330,307, filed on Apr. 30, 2010.
(60) Provisional application No. 61/178,015, filed on May 13, 2009.

(51) Int. Cl. A61M 5/36

(52) U.S. Cl. 604/122

(57) ABSTRACT

A laminar valve flow module is disclosed and may include a proximal chamber, a purge chamber, and a sheath chamber in fluid communication with each other and between an inlet opening and an exit opening. A guide chamber may be located within the proximal chamber and in fluid communication with the inlet opening, the proximal chamber, and the sheath chamber. The sheath chamber is in fluid communication with the exit opening.

### CROSS-REFERENCE TO RELATED APPLICATIONS

This application claims the benefit of the filing date of the pending provisional application entitled “LAMINAR VALVE FLOW MODULE”, filed Apr. 30, 2011, Ser. No. 61/330,307. This application is also a continuation in part of the pending nonprovisional application entitled “TRANS-SEPTAL ACCESS SYSTEM”, filed May 13, 2010, Ser. No. 12/779,951, which claims priority to the provisional application entitled “TRANS-SEPTAL ACCESS SYSTEM”, filed May 13, 2009, Ser. No. 61/178,015. The disclosures of all of the foregoing applications are hereby entirely incorporated herein by reference.

### BACKGROUND

1. Technical Field

This document relates to a laminar valve flow module.

2. Background

During certain cardiac procedures, catheters or other devices may be inserted into a patient's vascular system and pushed through blood vessels to reach a desired location. Once the desired location has been reached, the tissue at that location may be treated using any of a variety of devices. For example, treatment of certain cardiac arrhythmias which occur when contraction initiating signals originate within one or more of the pulmonary veins rather than at the sino-atrial node (SA) may include the introduction of a catheter into the left atrium of the patient to form a conduction block between the source of the improper contraction initiating signals and the left atrium.

In many such procedures, cardiac sheaths are used to facilitate insertion and exchange of the devices used to treat the affected tissue. These cardiac sheaths are tubes that are inserted into patients' vascular systems to act as guides for the other devices. For example, the distal end of a cardiac sheath may be inserted into a patient's femoral vein and advanced to the site to be treated with an open proximal end thereof remaining accessible from outside the patient. A catheter or other device may then be inserted through the sheath, which guides the device into the vascular system. If a first device needs to be replaced with a second, the first device is withdrawn from the sheath and the second device is inserted therethrough.

When a device is inserted through the sheath, air may be carried into the sheath with the device. This air may form bubbles, or emboli, when entering the blood stream, preventing normal blood flow to the heart and brain and potentially causing tissue damage or death of the patient. In particular, if devices used in treating the patient must be exchanged repeatedly via a sheath, great care must be exercised to prevent formation of emboli. Furthermore, the leakage of blood from such a sheath must be prevented while allowing insertion and refraction of devices there through.

### SUMMARY

Aspects of this document relate to a laminar valve flow module which prevents the introduction of air into a patient during the insertion of a medical device into the vasculature thereof, all the while indicating the direction of curvature of the medical device. These aspects may comprise, and implementations may include, one or more or all of the components and the like set forth in the appended DRAWINGS and CLAIMS, which are hereby incorporated by reference.

Particular implementations may include one or more or all of the following features, characteristics, benefits, advantages, etc.

Any laminar valve flow module disclosed in this document may at least in part be an air block for industrial, medical, and non-medical uses, such as during access to vessels, chambers, canals or containers, or for medical purposes such as during access to the cardiovascular system or other body vessels or lumens, especially procedures performed in the fields of cardiology, radiology, electrophysiology, and surgery. For example, laminar valve flow module implementations may be coupled or removably coupled to the proximal end of a vascular access sheath and introducer.

Laminar valve flow module implementations may provide a fluid barrier that isolates the inserted mapping or therapy catheter from outside air during transseptal procedures and the sheath during transseptal crossing. Thus, they may further prevent air from entering the introducer and provide for removal of the air or other gas from their chambers before it can enter the introducer where it could cause harm to a patient. Laminar valve flow module implementations may be attached to various standard proximal introducer terminations including Luer fittings and hemostasis valve outer barrels.

With laminar valve flow module implementations, air-free trans-septal crossing may be provided, which automatically prevents room air induction during left heart interventions. With laminar valve flow module implementations protection is user independent and automatic with a transparent body to provide the operator with a clear view of the fluid path. This provides the user with continuous viewing of fluid flow and a positive method to verify an air-free fluid environment. This also allows the user to eliminate air before it comes into contact with a patient. Thus, they are not position dependent and do not require continual monitoring. A user must overtly open the valve to allow air to enter.

Also, laminar valve flow module implementations may provide a scalable platform to accommodate current 8.5 French up to 21 French, to accommodate current AF mapping systems as well as large devices for valve replacement, as well as allow for employment of the Standard, Mullins Technique, using a Brockenbrough needle. They are scalable to catheter size and to the flow rate desired. Furthermore, laminar valve flow module implementations may provide a Hemostatic valve that closes over 0.014″ guidewire, accommodates 0.050″ Brockenbrough needle to cross the septum, and/or accommodates a proprietary dilator with an integral stylet for initial fossa ovalis crossing.

Laminar valve flow module implementations may have a shaped distal sheath tip and a distal tip deflection from 0 through 360 degrees.

They may also allow for single hand manipulation consistent with current techniques. They provide in-line selection and control with laminar concentric, visual controlled flow monitoring.

Laminar valve flow module implementations permit introduction of other devices or instrumentation through themselves and on into a lumen of the introducer while minimizing fluid loss or gain into the introducer. This device path is both separate and distinct from and is allowed simultaneously with the fluid path, both paths being controlled independently from one another. Laminar valve flow module implementations may have an integral, built in rotatable valve to selectively isolate outside air from the device, while simultaneously providing isolation of chambers to permit purging, fluid aspiration, and continuous air-free laminar saline fluid flow through the device into the patient with or without a working catheter present.

Valve positions of laminar valve flow module implementations cannot be inadvertently changed. The operator may position and set the modules and not have to worry about inadvertent changes.

Laminar valve flow module implementations may have a working catheter guide tube extending between internal hemostasis valves formed with corkscrew-like slots or angular slots at 45° to the long axis of guide to expel air induced by working catheter insertion from the catheter path, as well as a pliable proximal chamber capable of being squeezed and propelling fluid from a supply bag and, in the process eliminating air from the device, either purging or introducing working catheters into the body during an interventional procedure. This pliable proximal chamber provides a feeling of familiarity (e.g., approximates the feeling of an IV bag).

Laminar valve flow module implementations may also have self-aligning ports to introduce fluid into the system and to allow purging of air, fluid or blood. When disconnected, these valves prevent air from entering the system or allowing fluid to leak from the system.

Moreover, laminar valve flow module implementations do not increase drag on catheters. They also may prevent accidental detachment during long procedures, and provide 1:1 torque, no whipping (easy rotation, steering gear/tip position indicator). Laminar valve flow module implementations may also be integral to a sheath to prevent capture of air during attachment to sheath.

The foregoing and other aspects, features, and advantages will be apparent to those of ordinary skill in the art from the DESCRIPTION, DRAWINGS, and CLAIMS.

### BRIEF DESCRIPTION OF THE DRAWINGS

Implementations will hereinafter be described in conjunction with the appended DRAWINGS (which are not necessarily to scale), where like designations or elements denote like elements; and

FIG. 1 is a top perspective view of a laminar valve flow module implementation;

FIG. 2 is a top view of the laminar valve flow module implementation of FIG. 1;

FIG. 3 is a cross-sectional view of the laminar valve flow module implementation of FIG. 1 taken along line 3-3 of FIG. 2;

FIG. 4 is an exploded top perspective view of the laminar valve flow module implementation of FIG. 1;

FIG. 5 is a top perspective and hidden line view of a valve bobbin half of the laminar valve flow module implementation of FIG. 1;

FIG. 6 is a top perspective and hidden line view of a main valve body of the laminar valve flow module implementation of FIG. 1;

FIGS. 7-11 are front, side, top perspective, top, and cross-sectional views, respectively, of the laminar valve flow module implementation of FIG. 1 during a working mode of operation;

FIGS. 12-16 are front, side, top perspective, top, and cross-sectional views, respectively, of the laminar valve flow module implementation of FIG. 1 during an infuse/inject/aspirate mode of operation;

FIGS. 17-21 are front, side, top perspective, top, and cross-sectional views, respectively, of the laminar valve flow module implementation of FIG. 1 during a prime/purge mode of operation;

FIGS. 22-24 are front, side, and top perspective views, respectively, of the laminar valve flow module implementation of FIG. 1 with its strain relief and distal control member or hub in an aligned position;

FIGS. 25-27 are front, side, and top perspective views, respectively, of the laminar valve flow module implementation of FIG. 1 with its strain relief and distal control member or hub in a right or clockwise rotated position;

FIGS. 28-30 are front, side, and top perspective views, respectively, of the laminar valve flow module implementation of FIG. 1 with its strain relief and distal control member or hub in a left or counter-clockwise position;

FIGS. 31-32 are front and top perspective views, respectively, of the laminar valve flow module implementation of FIG. 1 with its sheath tip in an aligned position;

FIGS. 33-34 are front and top perspective views, respectively, of the laminar valve flow module implementation of FIG. 1 with its sheath tip in a right or clockwise rotated position; and

FIGS. 35-36 are front and top perspective views, respectively, of the laminar valve flow module implementation of FIG. 1 with its sheath tip in a left or counter-clockwise position.

### DESCRIPTION

This document features laminar valve flow module implementations which prevent the introduction of air into a patient during the insertion of a medical device into the vasculature thereof. Laminar valve flow module implementations may include a device inlet opening and an exit opening which allow the distal end of a medical device to pass through a rotatable guide chamber before it enters a sheath coupled to the exit opening. There are many features of laminar valve flow module implementations disclosed herein, of which one, a plurality, or all features or steps may be used in any particular implementation.

In the following description, reference is made to the accompanying DRAWINGS which form a part hereof, and which show by way of illustration possible implementations. It is to be understood that other implementations may be utilized, and structural, as well as procedural, changes may be made without departing from the scope of this document. As a matter of convenience, various components will be described using exemplary materials, sizes, shapes, dimensions, and the like. However, this document is not limited to the stated examples and other configurations are possible and within the teachings of the present disclosure.

In accordance with current terminology pertaining to medical devices, the proximal direction will be that direction on the device that is furthest from the patient and closest to the user, while the distal direction is that direction closest to the patient and furthest from the user. These directions are applied along the longitudinal axis of the device, which is generally an axially elongate structure having one or more chambers or channels extending through portions of the device. Some may extend from the proximal end to the distal end and possibly run substantially the entire length of the device. A sheath is an axially elongate tube that can also be termed a catheter, a cannula, an introducer, or the like.

Overview

During certain interventional procedures that require vascular access, a patient is catheterized through a vein or artery and a device is routed to the heart or other region of the cardiovascular system. The initial steps involve placement of a hollow tube within the blood vessel. The hollow tube can be a sheath or catheter for example. In many cases, these sheaths are fairly long.

Typical arterial catheter procedures include percutaneous transluminal coronary angioplasty, coronary stenting, aortic stent-graft procedures, endarterectomy, and the like. Introducers, catheters or other devices are routinely routed through these sheaths into the arterial side of the circulatory system where pulsatile blood pressure generally averages 100 mm Hg cycles and pulses at an average rate of approximately 1 to 3 beats per second. The peak systolic pressures in the arterial side in a normal patient are around 110 to 130 mm Hg and the lowest diastolic pressures are around 70 to 90 mm Hg. In a hypertensive patient experiencing what is known as high blood pressure, the peak systolic arterial pressure can exceed 250 mm Hg. A catheterization lab or operating room is typically a clean room, which is maintained at positive pressure ranging from 0 to 2 mm Hg. When a catheter is routed into the arterial system, the distal end of a through lumen will be exposed to these arterial blood pressures and a positive pressure gradient will exist between the distal end and the proximal end of the catheter can be such that, unless proper hemostasis is maintained, blood is forced out through the catheter into the ambient environment.

The number of venous procedures being performed each year is increasing as more endovascular therapies evolve or are developed for pathologies such as atrial fibrillation, mitral valve repair, mitral valve replacement, and the like. Introducers, catheters or other devices are also routed through sheaths into the venous side. Its distal end is exposed to central venous blood pressure, which cycles at the same rate as the arterial side, approximately 1 to 3 beats per second. The normal, healthy, pulsatile venous pressures are lower than those in the arterial side and can range between low values of around 3 to 5 mm Hg and peak values of around 15 to 20 mm Hg with an average of approximately 10 mm Hg.

In the central venous circulation, for example, as measured in the right atrium of the heart, the distal end of the sheath can be exposed, during part or all of the cardiac cycle, to pressures equal to or below those to which the proximal end of the sheath is exposed. When the room or ambient pressure, to which the proximal end of the sheath is exposed, is above that of the distal end of the sheath, a negative pressure gradient or pressure drop can occur. Such a negative pressure drop allows air to be forced into the proximal end of the catheter. Should the air reach the distal end of the catheter by way of a through lumen, it could escape into the blood stream in the form of large or small bubbles, resulting in an air embolism. Such air embolisms can cause harm to the health of the patient, or even death, and need to be avoided.

This situation can be exacerbated by ambient room pressures often found in the environment of the clean room, operating theatre, or catheterization lab. Under normal conditions, the environment of the clean room, operating theatre, or catheterization lab can be maintained at an elevated air pressure of around 5 to 10 mm Hg above exterior air pressure. Thus, a right atrial pressure, which momentarily dips to 2 mm Hg, can be overcome by a room air pressure of 2 to 3 mm Hg causing air to be forced retrograde through the catheter and into the circulatory system.

During a venous procedure and in preparation for a trans-septal puncture, the distal end of the catheter can reside in the vena cava or right atrium for a substantial amount of time. Such positioning renders the catheter at risk for being exposed to a negative pressure drop and the potentially catastrophic consequences of retrograde air flow. An air embolism or bubble escaping into the venous circulation can lodge in the lungs causing a pulmonary embolism. Left sided (arterial) procedures, which are accessed from the right (or venous) side present a further complication in that a gas bubble or embolism that escapes into the arterial side can be pumped by the heart to sensitive tissues where it can lodge, prevent distal blood flow, and thus cause ischemia. Such ischemia is potentially life threatening if it occurs in the cerebrovasculature or the coronary arteries.

Instances can arise where a hemostasis valve breaks or becomes disconnected from the sheath or catheter and a substantial bolus of air can enter the cardiovascular system with sometimes catastrophic consequences. Even without such equipment failure, operator error can result in air being pumped retrograde into the blood stream by ambient air pressure, if a Tuohy-Borst valve is not properly adjusted, a hemostasis valve becomes distorted, or too small a catheter is used for the type of hemostasis valve.

Structure

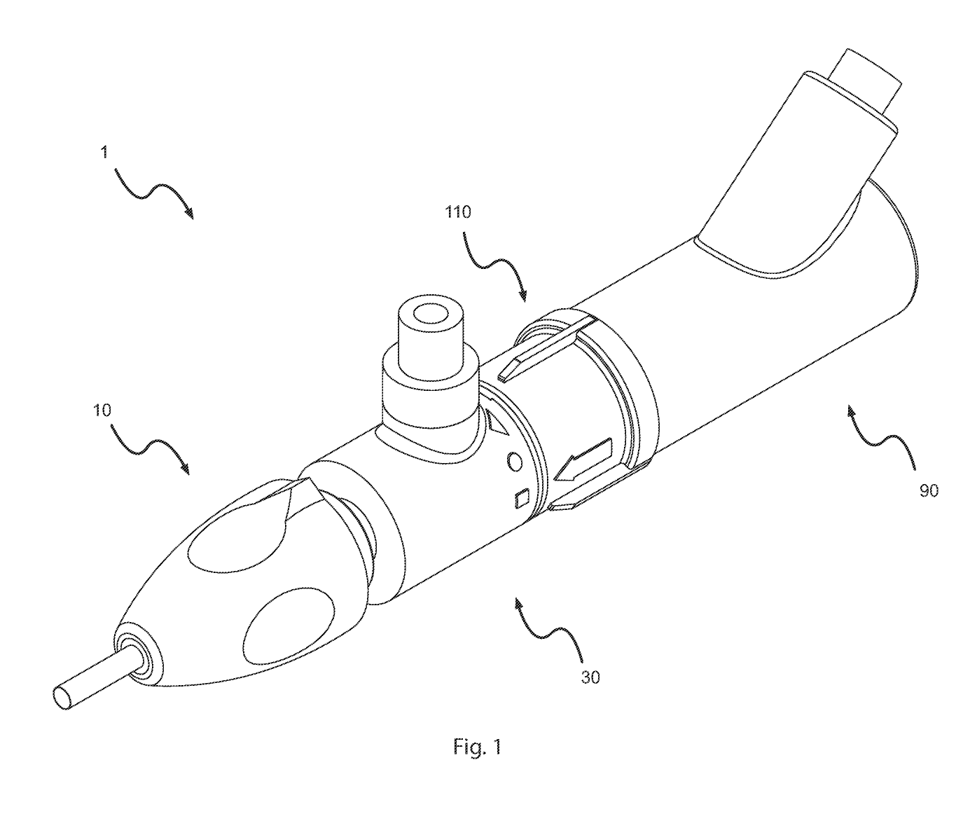

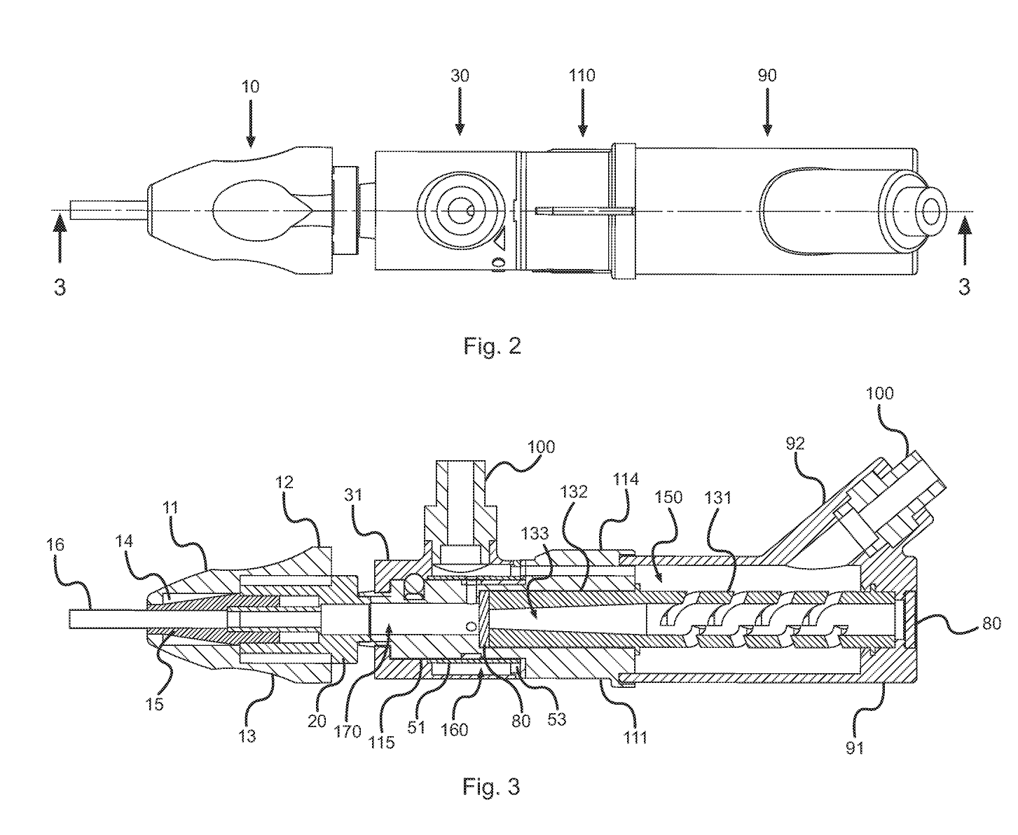

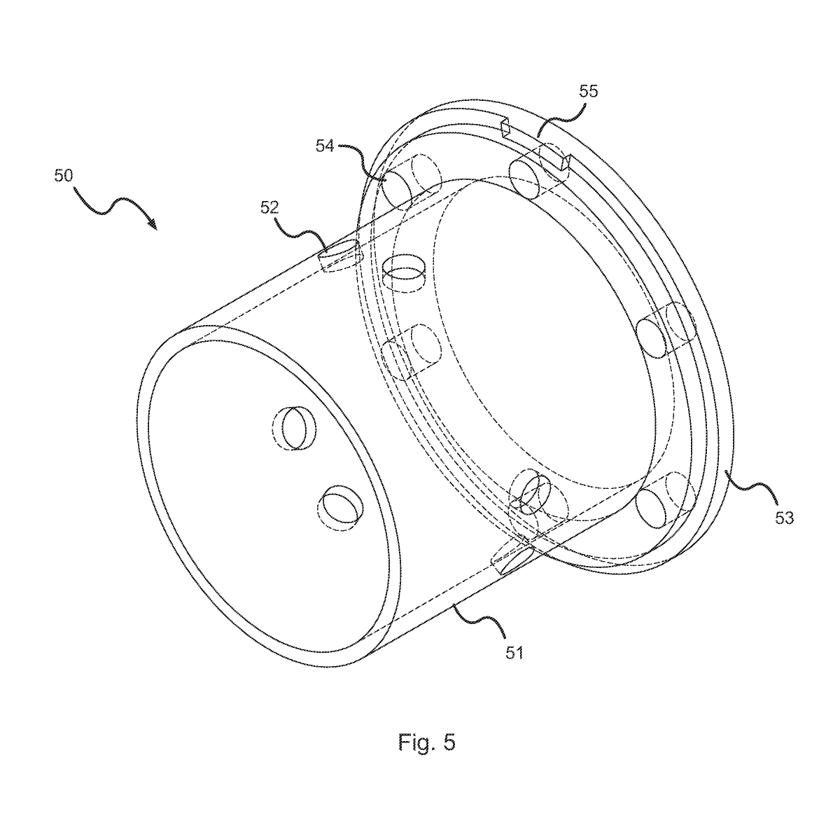

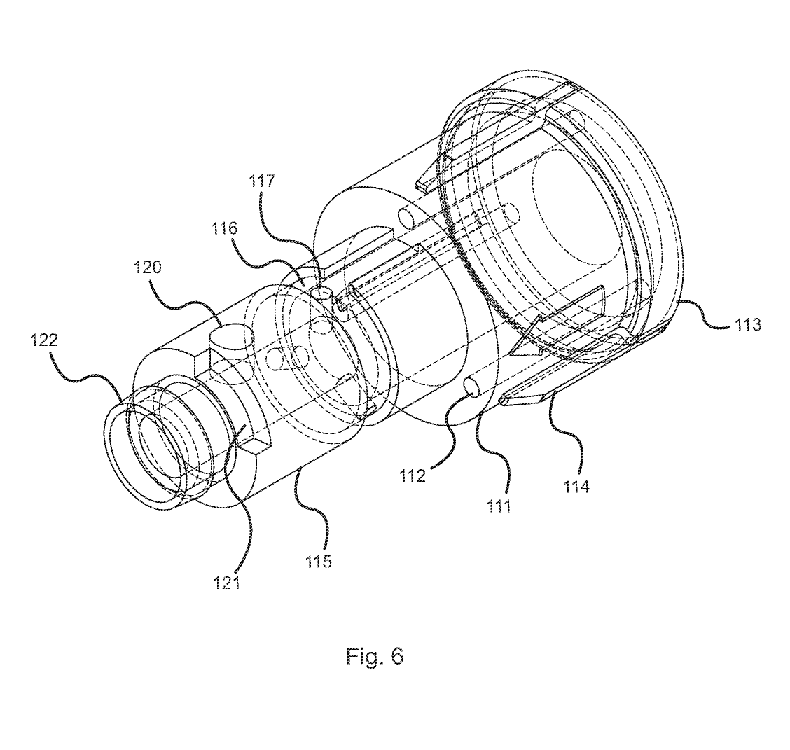

There are a variety of laminar valve flow module implementations. Notwithstanding, turning to FIGS. 1-6 and for the exemplary purposes of this disclosure, laminar valve flow module 1 is shown and described. Laminar valve flow module 1 provides for both a valve function as well as a steering function making it very appropriate for atrial access and maneuvering into the left ventrice for example.

Laminar valve flow module 1 combines components together to make a stronger device. With such a reduced number of total parts, the overall cost to manufacture is lowered. In addition, molded-on ports (designed to accept the Qosina bi-directional check valve with molded-on luer lock for example) have also been included. Furthermore, an integral introducer access sheath with a high-pressure rotatable connector that cannot detach during either trans-septal crossing or the mapping/ablation procedure may be included, as well.

Laminar valve flow module 1 is designed to provide an air and fluid-tight port for entry into the venous system of the body and at the same time allow working medical devices to be inserted. Laminar valve flow module 1 is dual-valve fluid chamber that provide hemostasis during catheter introduction and/or exchange and venting of any entrained air by the catheter. Through a series of chambers that can be sealed off from each other, fluid and gas can be directed through module 1 in a controlled manner. Located on module 1 are two ports, an input port and an output port. The output port contains a bi-directional, luer-actuated valve that provides for aspiration, blood sampling, and pressure monitoring. The input port contains a bi-directional, luer-activated valve to allow for fluid infusion.

Generally, laminar valve flow module 1 may include: strain relief and distal control member or hub assembly 10; high pressure Luer 20; straight ported bobbin half 30; two bi-directional check valves 100; valve bobbin half 50; tubular seal 60; ring seal 70; distal hemostasis valve 80; squeeze chamber seal port 90; main valve body with port 110; slotted guide-tube 130; and steel ball 118. Thus, laminar valve flow module 1 may comprise eight main components (elements 10, 20, 30, 100, 50, 10, 110, and 130), three seals (elements 60, 70, and 80), and one integrated flexible body and seal (element 90) that form module 1 and that define three chambers for directing fluid and gas flow, namely proximal chamber 150, purge chamber 160, and distal sheath chamber 170. Various holes are formed and defined into main valve body with port 110 and valve bobbin half 50, and their interaction and alignment or not allows for controlling the fluid and gas path inside of module 1.

The relationships between the components are shown and described in the appended DRAWINGS and their functions are described throughout this document.

Notwithstanding, strain relief and distal control member or hub 10 may include a body 11. Body 11 may be cone shaped. Defined on a surface of body 11 may be at least one recess 13 configured to aid a user's thumb or finger in gripping hub 10. Body 11 may also define on a surface at its proximal end portion a tactile indicator for the user (e.g., a protruding ridge 12) that indicates (by its position as the distal rotating hub 10 is rotated) the direction of curvature of the medical device (the direction of catheter tip 17 orientation). Body 11 also defines a stepped or two tiered internal through hole or cavity 14 and a distal end that defines an opening smaller than cavity 14.

Sheath 16 has hub 15 coupled at its proximal end. Hub 15 is located within the smaller tier or step of cavity 14 and opening in the distal end of body 11 such that sheath 16 extends out distally from hub 10. The proximal end of hub 15 is coupled and in fluid flow communication with the distal end of Luer 20 around hollow through tube 22 inside cavity 21.

Distal Luer 20 (e.g., Qosina 80353) may be a rotating Luer that allows for high pressure connections. Alternatively, Luer 20 could lock in place so that a working catheter, for example, can use it as a platform for redirection. It may lock in place via a ratcheting mechanism (e.g. meshing gears) or through a detent mechanism for example.

Distal rotating Luer 20 and hub 10 can rotate an associated medical device (e.g. sheath 16, a catheter, etc.) within a 360 degree range, thereby providing strain relief and steering capabilities. The distal rotating hub 10 may be overmolded onto the rotating Luer 20 (Luer 20 located in the proximal larger tier or step of cavity 14 and abutting the shoulder transition in cavity 14) so that they are not removable from one another (fixedly attached). This ensures that they cannot be disassembled and that everything remains sealed so no air can enter module 1.

A rotating valve assembly may be able to be rotated to any number of positions (e.g., three positions as shown). The valve assembly rotates freely and independent from hub 10 and vice versa. The valve assembly includes straight ported bobbin half 30 and valve bobbin half 50 that rotate around main valve body with port 110.

Straight ported bobbin half 30 (one half of purge chamber 160 for evacuation of air, fluid, and blood) may be formed of an acrylic material. The proximal end of Luer 20 is rotatingly coupled and in fluid flow communication with the distal end of bobbin half 30 through its distal opening. Bobbin half 30 includes a body 31 that defines port 32 coupled to bidirectional check valve 100 (e.g., Qosina 80121). Bobbin half 30 also defines index markings on a surface at the its proximal end portion, namely triangle 33, circle 34, and square 35. Bobbin half 30 also defines an internal cavity into which valve bobbin half 50 is coupled and in fluid flow communication.

Valve bobbin half 50 (the other half of purge chamber 160 for evacuation of air, fluid, and blood) includes a radial flange or face 53 and tubular body 51 formed of an acrylic material. Face 53 defines at least one through hole (e.g., three axial through holes 54 as shown) and tubular body 51 defines at least one through hole (e.g., three radial through holes 52 as shown). Tubular body 51 couples and is fluid flow communication with the internal cavity of bobbin half 30 and face 53 abuts the proximal end of bobbin half 30.

Main valve body with port 110 is a valve body with axial flow holes and radial ports for fluid access. Main valve body 110 includes at least one through hole (e.g., three axial through holes 112 as shown) through its body portion 111 and at least one through hole (e.g., three radial through holes 117 as shown) around its circumference in seal seat 116. Body 111 also includes ridges 114, index marking arrow 123, and a proximal face 113. Body 115 defines a radial surface slot 121 at its distal end portion and hole 120 that receives a ball and detent assembly that includes steel ball 118 and resilient member 119. Body 115 also defines Luer connection member 122 at its distal end. Luer connection member 122 both couples to (snaps in) and extends through the distal opening of bobbin half 30 to couple with Luer 20 and to retain main valve body 110 and bobbins 50 and 30 together. Main valve body 110 also defines an internal cavity into which distal hemostasis valve 80 and slotted guide-tube 130 are coupled and in fluid flow communication.

Tubular seal 60 (slotted seal made of silicone) and ring seal 70 (slotted seal made of silicone) may be coupled together or integral with one another and seated in or molded in seat 116.

Dual hemostasis valves namely distal hemostasis valve 80 and proximal hemostasis valve 80 are included. Distal hemostasis valve 80 is located close to the holes in the valve assembly to avoid clots and prevents blood from a patient inadvertently coming back into proximal chamber 150 and clouding fluid so bubbles cannot be visualized. Proximal hemostasis valve 80 is integrated into or molded into the proximal end of squeeze chamber 90.

Squeeze chamber seal port 90 may be formed of a vinyl material and may include a pliable body 91. Chamber 90 can be clear and can be squeezed. Such a clear and pliable chamber 90 provides a feeling of familiarity, eliminates parts by allowing integration of proximal hemostasis valve 80, allows the visualization of air bubbles that can be isolated from the patient and drawn off, all the while propelling fluid from a supply bag and, in the process eliminating air from module 1, either purging or introducing working catheters into the body during an interventional procedure. Pliable body 91 includes a built-in/integral hemostasis valve 80 and a built-in/integral flexible strain relief connection 92 coupled to bidirectional check valve 100 (e.g., Qosina 80121). Alternatively, proximal chamber 90 could be rigid depending on user preference.

Slotted guide-tube 130 is a guide tube for directing dilator/catheter/guide wire through module 1. Guide-tube 130 may include a guide chamber 133 that may be tapered to help guide medical devices through distal hemostasis valve 80 to which its distal end abuts or is coupled to. Guide-tube 130 may also include slotted chamber 131 may also define corkscrew-like slots along or angular slots at 45 degrees to its long axis to expel air induced by working catheter insertion from the catheter path. Slots configured in such a manner help ensure that no bubble can travel over the length of the path. Such slots are also more conducive to linear catheter travel.

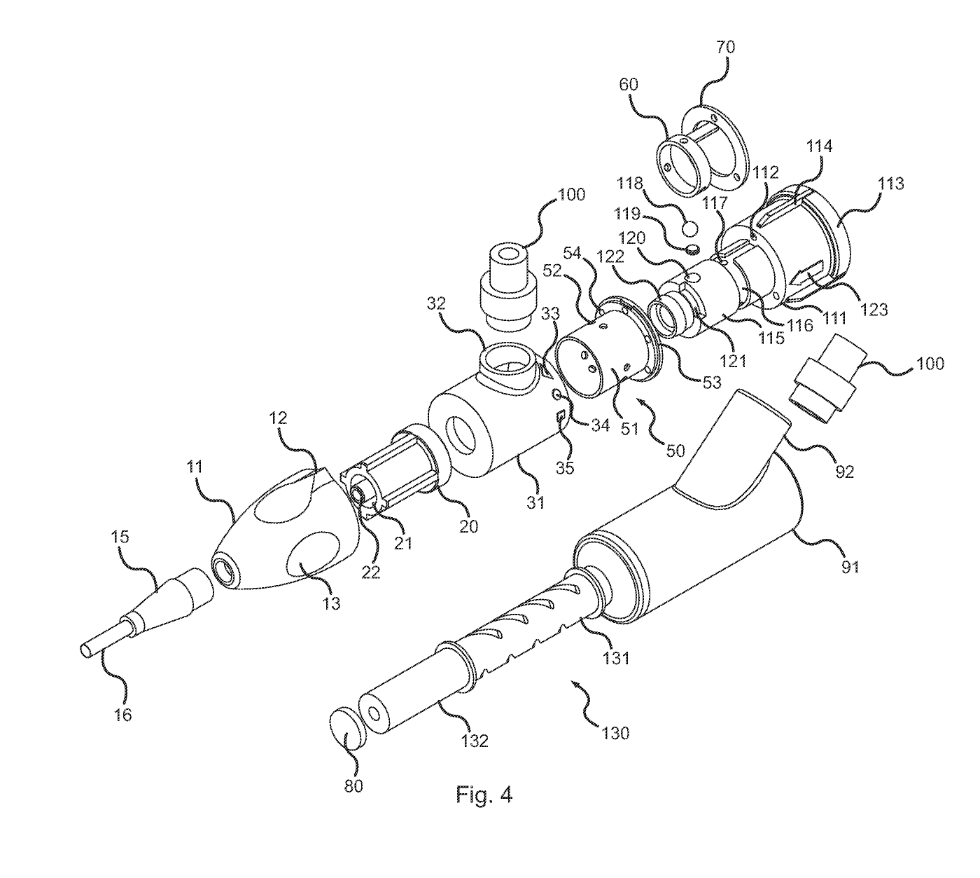

Thus, when module 1 is assembled as depicted in FIGS. 3-4, infusion or “in” port 92 and proximal bidirectional check valve 100 connected to flexible body 90 will allow fluid and gas into proximal chamber 150 formed by valve bobbin half 50, tubular seal 60, ring seal 70, distal hemostasis valve 80, squeeze chamber seal port 90, main valve body with port 110, and slotted guide-tube 130. Purge chamber 160, formed by straight ported bobbin half 30, distal bidirectional check valve 100, valve bobbin half 50, and main valve body with port 110, can be sealed from proximal chamber 150 and distal sheath chamber 170 by tubular seal 60, ring seal 70, and distal hemostasis valve 80, and will allow fluid and gas to flow out through purge/infuse or “out” port 32 and distal bidirectional check valve 100 connected to straight ported bobbin half 30. Distal sheath chamber 170 is formed by hub 10, high pressure Luer 20, straight ported bobbin half 30, distal bidirectional check valve 100, valve bobbin half 50, distal hemostasis valve 80, and main valve body with port 110. Fluid and gas will be able to flow out of the end module 1.

Other Implementations

Many additional implementations are possible.

For the exemplary purposes of this disclosure, laminar valve flow module implementations may be included in a kit. For example, such a kit may include: one or two sheaths and dilators; 0:032″ Guidewire; a Brockenbrough needle; and/or IFU (non-slerile).

The sheath and dilator are radiopaque to enable visualization under fluoroscopy and have a specially curved distal portion to accommodate positioning against the atrial septum and to accommodate a 0.032″ guidewire and a Brockenbrough type curved puncture needle. The dilator is tapered at the distal tip with an internal lumen designed to accept ancillary devices (e.g., needles or guidewires) that have a maximum diameter of 0.032″. The inner-lumen of the dilator is also designed with a special geometry (internally tapered) at its distal end to limit the exposure of the Brockenbrough needle (to capture the outer body of the Brockenbrough needle, thus allowing only the needle to project beyond the dilator tip). Each introducer features vent holes (e.g., four holes) to keep fluid flow around the tip to reduce cavitation during aspiration and device withdrawal and to allow purging (suction) even when the tip is blocked by the working catheter or cardiac tissue for example.

Such a kit may be packaged in a Tyvek Bag with or without a card, or in a tray with a peel away Tyvek cover. Blocks may be included in the tray to prevent contents from “jumping” out as the cover is removed. The contents in the bag or tray may be sterile and may include enough components for one or two transseptal puncture crossing(s). The IFU may be attached to the tray or bag to be accessed prior to opening the tray or bag.

Further implementations are within the CLAIMS.

Specifications, Materials, Manufacture

It will be understood that laminar valve flow module implementations are not limited to the specific assemblies, devices and components disclosed in this document, as virtually any assemblies, devices and components consistent with the intended operation of a laminar valve flow module implementation may be utilized. Accordingly, for example, although particular assemblies, devices and components are disclosed, such may comprise any shape, size, style, type, model, version, class, measurement, concentration, material, weight, quantity, and/or the like consistent with the intended operation of a laminar valve flow module implementation. Implementations are not limited to uses of any specific assemblies, devices and components; provided that the assemblies, devices and components selected are consistent with the intended operation of a laminar valve flow module implementation.

Laminar valve flow module implementations and their components may be formed of any of many different types of materials or combinations thereof that can readily be formed into shaped objects provided that the materials selected are consistent with the intended operation of a laminar valve flow module implementation. For example, as disclosed above, or in addition or in lieu of materials disclosed above, the components may be formed of: rubbers (synthetic and/or natural) and/or other like materials; glass and/or other like material; polymers such as thermoplastics (such as ABS, Fluoropolymers, Polyacetal, Polyamide; Polycarbonate, Polyethylene, Polypropylene (low or high density), Polysulfone, Polyvinyl Chloride, Acrylic, Vinyl, Polystyrene, and/or the like), thermosets (such as Epoxy, Phenolic Resin, Polyimide, Polyurethane, Silicone, and/or the like), any combination thereof, and/or other like materials; carbon-fiber, aramid-fiber, any combination thereof, and/or other like materials; composites and/or other like materials; any other suitable material; and/or any combination of the foregoing thereof.

For the exemplary purposes of this disclosure, the chambers and other components may be fabricated from materials that are transparent and optically clear with a minimum of defects or blemishes. As such, bubbles can be visualized or identified by the user more easily so they can be removed or guided out.

Laminar valve flow module implementations may be manufactured using conventional procedures as added to and improved upon through the procedures described here. Some components defining laminar valve flow module implementations may be manufactured simultaneously and integrally joined with one another, while other components may be purchased pre-manufactured or manufactured separately and then assembled with the integral components. Accordingly, manufacture of these components separately or simultaneously may involve extrusion, vacuum forming, injection molding, blow molding, casting, forging, cold rolling, milling, drilling, reaming, turning, grinding, stamping, pressing, cutting, bending, welding, soldering, hardening, riveting, punching, plating, and/or the like. Components manufactured separately may then be coupled or removably coupled with the other integral components, if necessary, in any manner, such as with adhesive, a weld joint, a fastener, washers, retainers, wrapping, tubing, wiring, any combination thereof, and/or the like for example, depending on, among other considerations, the particular material forming the components.

Use/Operation

Laminar valve flow module implementations not only prevent the loss of substantial amounts of blood during arterial procedures, but they also prevent air backflow into the sheath or catheter and into the patient through catheters routed into the venous circulation. Laminar valve flow module implementations accept catheters or instrumentation through themselves and close the seal around those catheters. Laminar valve flow module implementations close quickly when the inserted catheter is removed. Laminar valve flow module implementations prevent air passage retrograde back into the catheter while still maintaining device operability.

Laminar valve flow module implementations allow work in a medical environment wherein a pressure-differential is expected. They will prevent air from entering and/or the escape of blood or other body fluids when a high pressure system (defined as above atmospheric) is accessed by interventional techniques.

For example, such laminar valve flow module implementations may be affixed to the proximal end of a primary sheath intended for vascular access. The laminar valve flow module implementation may be provided integral to the primary sheath or permanently attached to the primary sheath. The laminar valve flow module implementation permits introduction of catheters or other instrumentation through the central lumen of the primary sheath. The laminar valve flow module implementation may further prevent the loss of blood when the distal end of the catheter is exposed to circulating blood, either in the arterial or venous system. The laminar valve flow module implementation traps substantially any air entrained into its interior, prevents the air from entering the through lumen of the primary catheter, and can shunt the air out of itself through an out port.

While certain implementations are described with respect to endovascular uses or a catheter, laminar valve flow module implementations are not so limited and can be configured for use in a variety of medical, non-medical and industrial uses where the blocking of gas is desired. For example, they may prevent gas from entering and/or the escape of gas of other materials from a vessel when a high pressure system is accessed. In implementations directed to industrial or non-medical uses, a wide variety of different types of ports may be used. Also, instead of a catheter, implementations may be adapted to receive a variety of devices such as tubular devices for insertion into containers, canals, vessels, passageways, or the like. Such devices can be designed, for example, to permit injection or withdrawal of fluids or to keep a passage open. For example, an implementation of the invention directed to industrial uses prevents gas from entering and/or the escape of gas of other materials from a vessel when a device is inserted into the vessel.

Laminar valve flow module implementations may comprise one way valves that permit flow only in a single direction and make sure that fluid can only flow in and that gas can only flow out. Accordingly, laminar valve flow module implementations may be operably connected to an external subsystem that provides a reservoir of liquid such as water, saline, Ringers solution, or the like pressurized to a level above that of the venous pressure. The fluid delivery subsystem is operably connected to the laminar valve flow module implementation by way of a tube, manifold, or the like. The laminar valve flow module implementation can also be operably connected to an external subsystem that withdraws or removes gas, specifically air, which can collect within the module. The gas removal subsystem is operably connected to the laminar valve flow module implementation. Although the subsystems are referred to as being external, they can also be internal, integral to, or affixed to the laminar valve flow module implementation. In an implementation, the gas removal subsystem can comprise a gas permeable membrane that permits gas such as air to pass but substantially prevents the loss of liquids such as water, saline, or blood. In this implementation, a pump is operably connected to withdraw the air out of the trap through the gas permeable membrane by generating a pressure drop within a range that facilitates such air passage.

In further describing the operation of laminar valve flow module implementations, and for the exemplary purposes of this disclosure, a laminar valve flow module implementation may be affixed to the proximal end of the primary catheter, cannula, introducer, or sheath. The primary catheter is flushed with saline and purged of air. The primary catheter is introduced into the vascular system, generally after first placing a guidewire, which is routed through the laminar valve flow module implementation. The laminar valve flow module implementation is connected to a source of normal saline. The laminar valve flow module implementation can also be connected to a fluid removal system. The primary catheter is routed to its target location. The secondary catheter, or catheters, can be inserted through the laminar valve flow module implementation and through the catheter lumen and into the vascular system at the target site. Any air that becomes entrained into the catheter guide chamber escapes through its slots and migrates into the proximal chamber and up to the top of the chamber. The trapped air either remains within the larger chamber or it is drawn off through an out port by the fluid removal system either into the air or into an air reservoir when the valve is in the appropriate position. The fluid removal system can be optimized to selectively withdraw only gasses such as air.

In further describing the operation of laminar valve flow module implementations, and for the exemplary purposes of this disclosure, laminar valve flow module 1 may have three general modes of operation. In the working mode of operation, fluid and gas are allowed to pass between proximal chamber 150, purge chamber 160, and distal sheath chamber 170 and into/out of module 1. In the infuse/inject/aspirate mode of operation, fluid and gas are allowed to pass only between purge chamber 160 and distal sheath chamber 170 and into/out of module 1. In the prime/purge mode of operation, fluid and gas are allowed to pass only between proximal chamber 150 and purge chamber 160 and into/out of module 1.

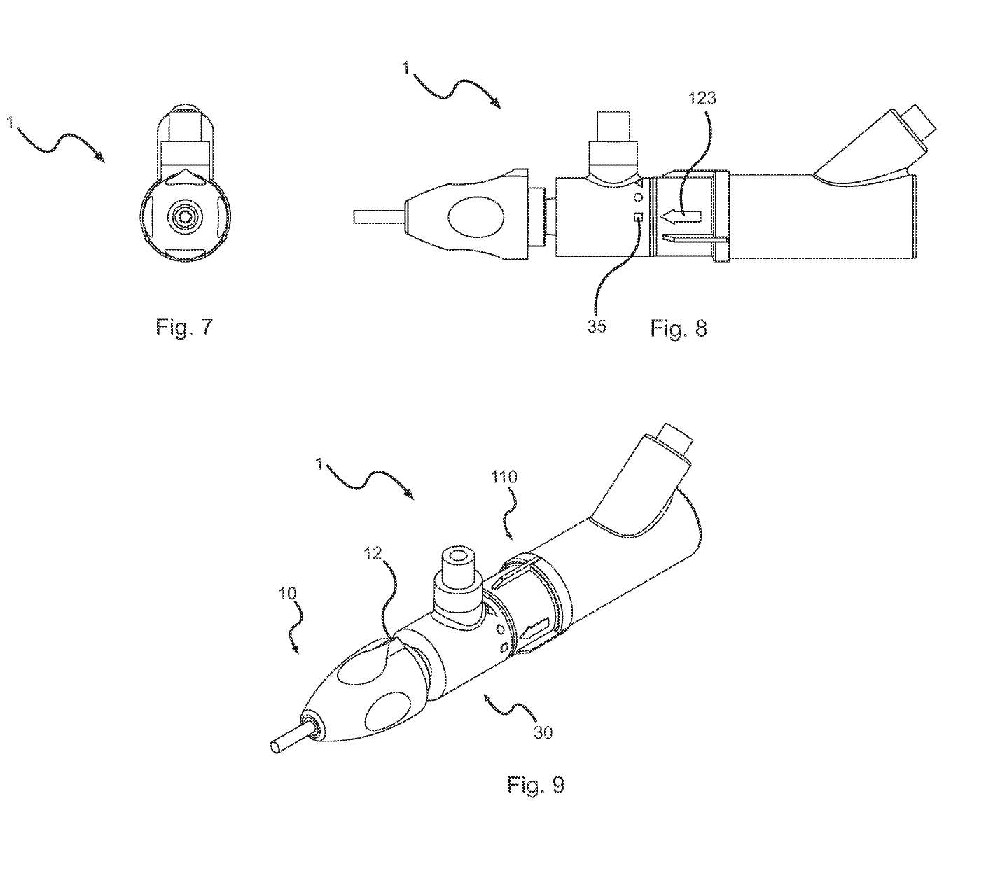

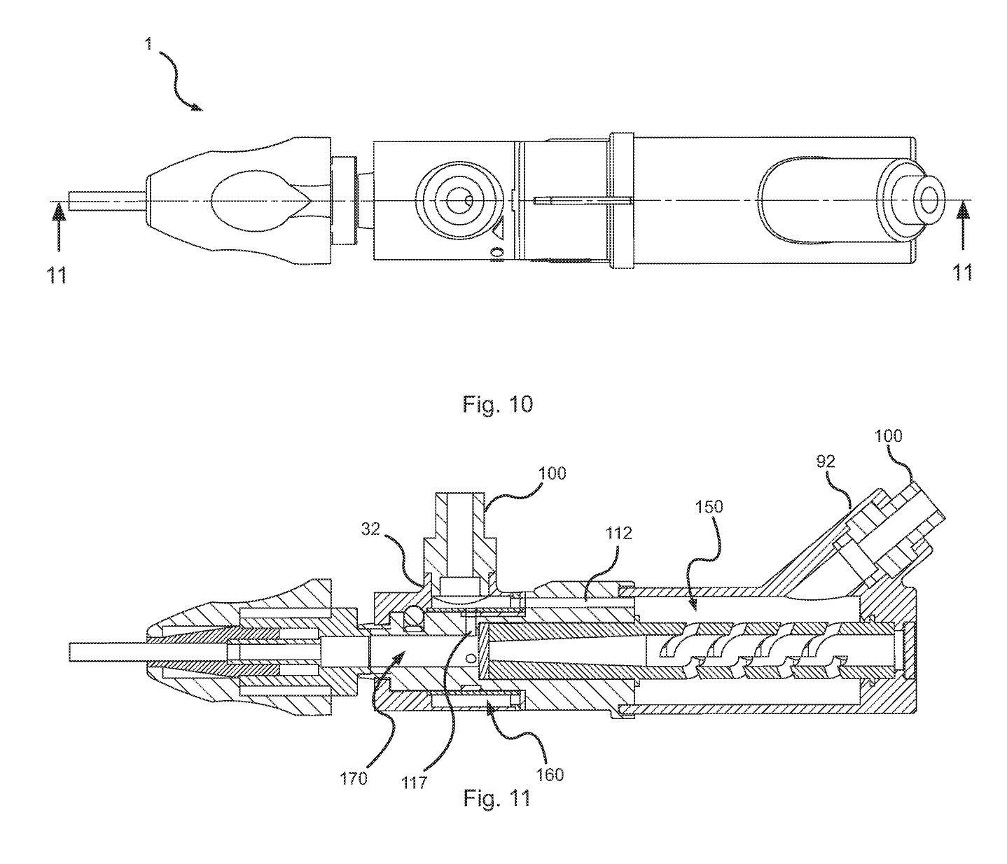

In the working mode of operation (FIGS. 7-11), all chambers are connected; fluid and gas are allowed to pass between proximal chamber 150, purge chamber 160, and distal sheath chamber 170 when straight ported bobbin half 30 and valve bobbin half 50 are rotated around main valve body with port 110 to align the pass-through holes 54 and 52 in valve bobbin half 50 and the corresponding pass-through holes in tubular seal 60 and ring seal 70 with the corresponding pass-through holes 112 and 117 in main valve body with port 110. Indexing marks (arrow 123 and square 35) will show that all the holes are in alignment. Note that hub 10 and its protruding ridge 12 do not move and rotate independent of the rotation of straight ported bobbin half 30 and valve bobbin half 50 around main valve body with port 110. Fluid and gas can be directed in or out of bidirectional check valve 100 and proximal port 92, in or out of distal bidirectional check valve 100 and distal port 32, or in or out of the distal end of module 1. Thus, fluid is allowed to flow through the entire module 1 even while a working catheter is inserted through module 1.

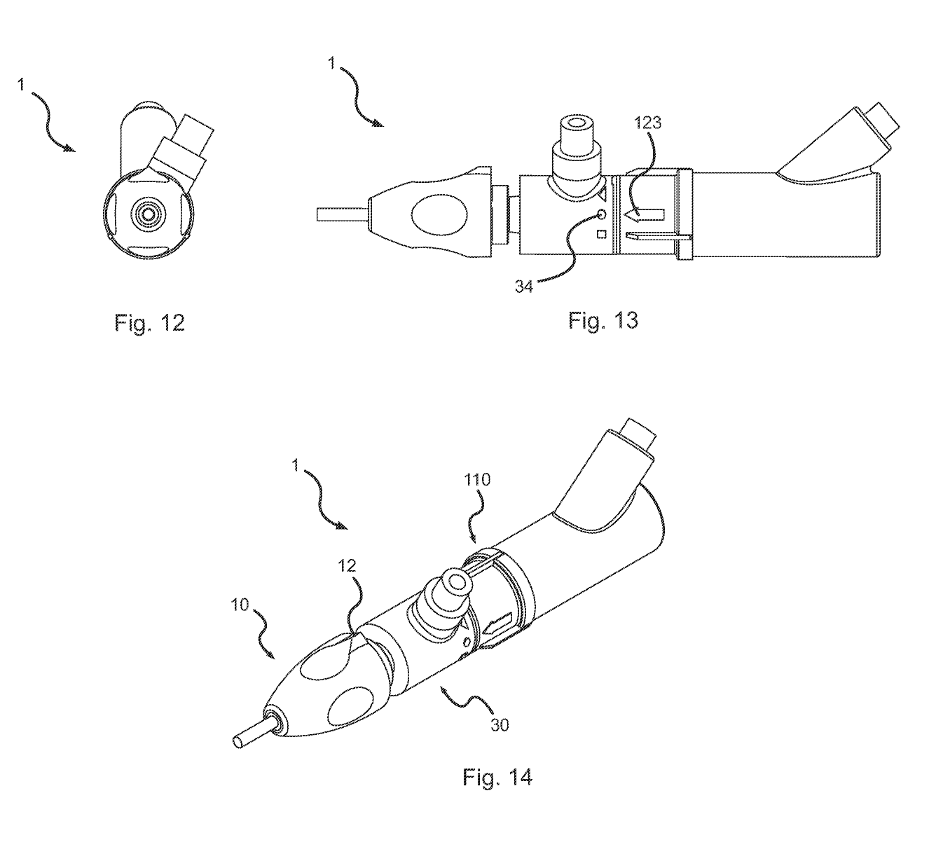

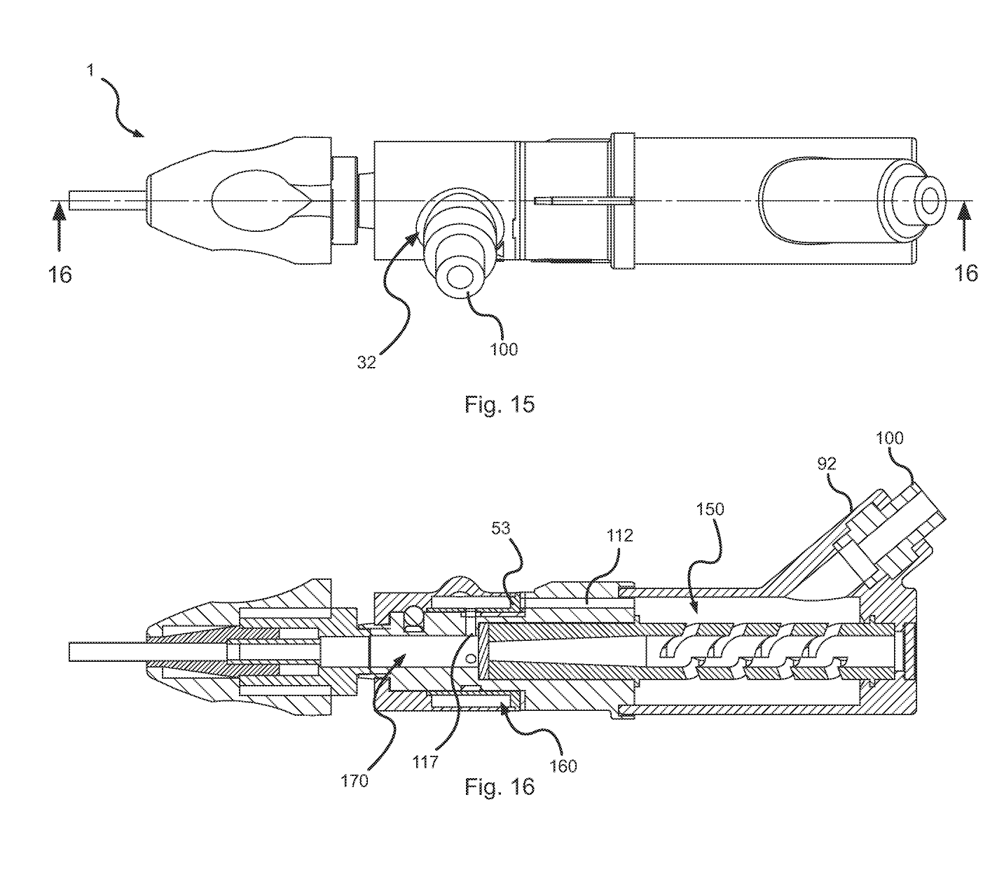

In the infuse/inject/aspirate mode of operation (FIGS. 12-16), fluid and gas are allowed to pass only between purge chamber 160 and distal sheath chamber 170 when straight ported bobbin half 30 and valve bobbin half 50 are rotated around main valve body with port 110 to align the pass-through holes 52 in valve bobbin half 50 with the corresponding pass-through holes in tubular seal 60 and pass-through holes 117 in main valve body with port 110. Indexing marks (arrow 123 and circle 34) will show that all the holes are in alignment. Note that hub 10 and its protruding ridge 12 do not move and rotate independent of the rotation of straight ported bobbin half 30 and valve bobbin half 50 around main valve body with port 110. Proximal chamber 150 and purge chamber 160 will be sealed off from each other due to the pass-through holes 54 in valve bobbin half 50 not being in alignment with the corresponding pass-through holes in ring seal 70 and pass-through holes 112 in main valve body with port 110. Fluid and gas can be directed from distal bidirectional check valves 100 and distal port 32 out through the open distal end of module 1 or fluid and gas can be directed from the open distal end of module 1 through distal bidirectional check valves 100 and distal port 32.

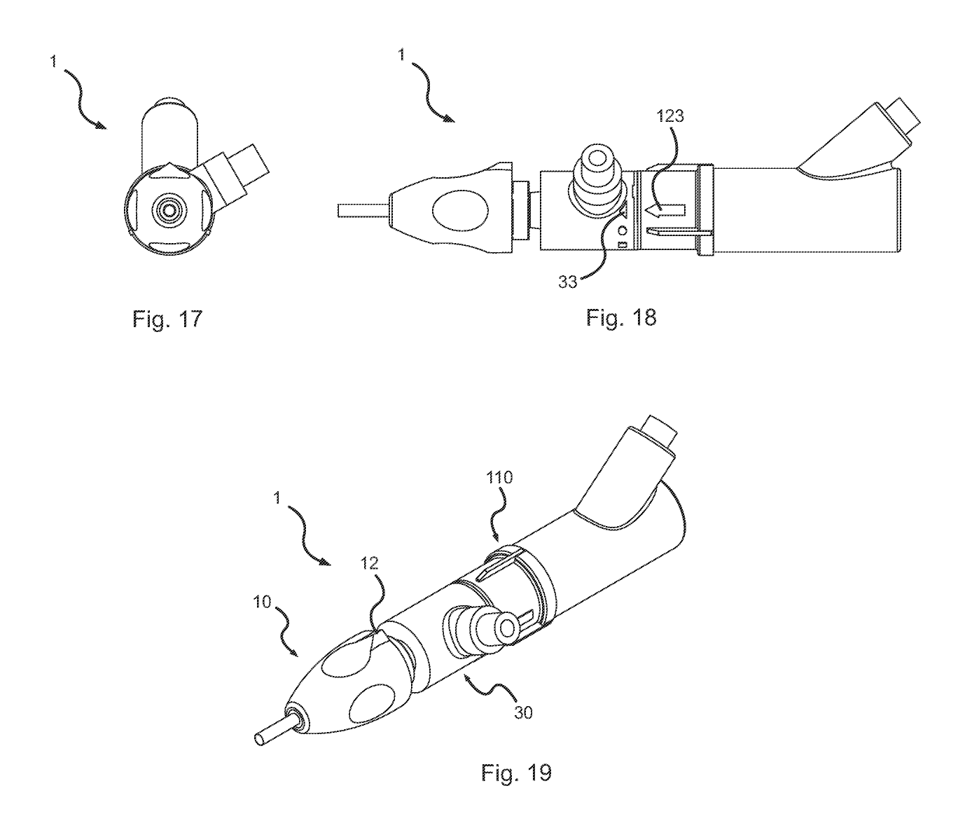

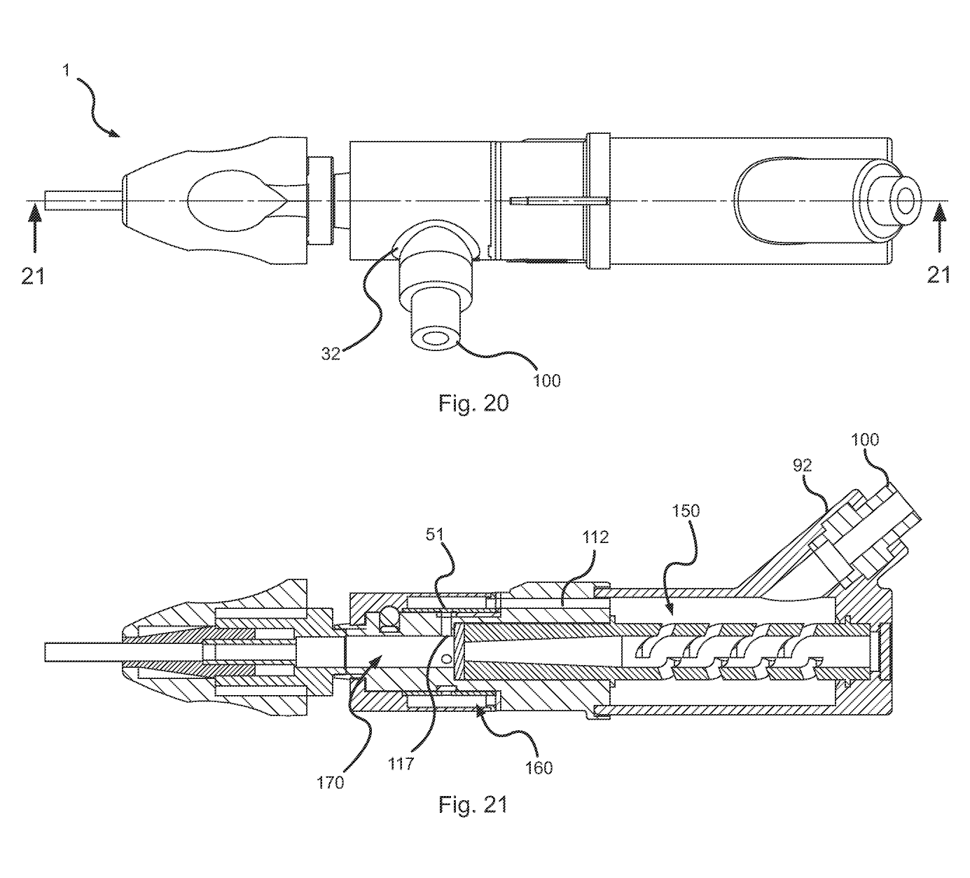

In the prime/purge mode of operation (FIGS. 17-21), fluid and gas are allowed to pass only between proximal chamber 150 and purge chamber 160 when straight ported bobbin half 30 and valve bobbin half 50 are rotated around main valve body with port 110 to align the pass-through holes 54 in valve bobbin half 50 and the corresponding pass-through holes in ring seal 70 and pass-through holes 112 in main valve body with port 110. Indexing marks (arrow 123 and triangle 33) will show that all the holes are in alignment. Note that hub 10 and its protruding ridge 12 do not move and rotate independent of the rotation of straight ported bobbin half 30 and valve bobbin half 50 around main valve body with port 110. Purge chamber 160 will be sealed off from distal sheath chamber 170 due to the pass-through holes 52 in valve bobbin half 50 not being in alignment with the corresponding pass-through holes in tubular seal 60 and pass-through holes 117 in main valve body with port 110. This will allow fluid and gas to pass from proximal chamber 150 to purge chamber 160. To help facilitate the passing of fluid and gas, squeeze chamber seal port 90 can be squeezed. Once the fluid and gas has entered into purge chamber 160, it can be evacuated through distal port 32 and distal bidirectional check valve 100.

In further describing the operation of laminar valve flow module implementations and for the exemplary purposes of this disclosure, the main components of laminar valve flow module 1 can operate independent of one another, which can be very useful during many procedures and techniques.

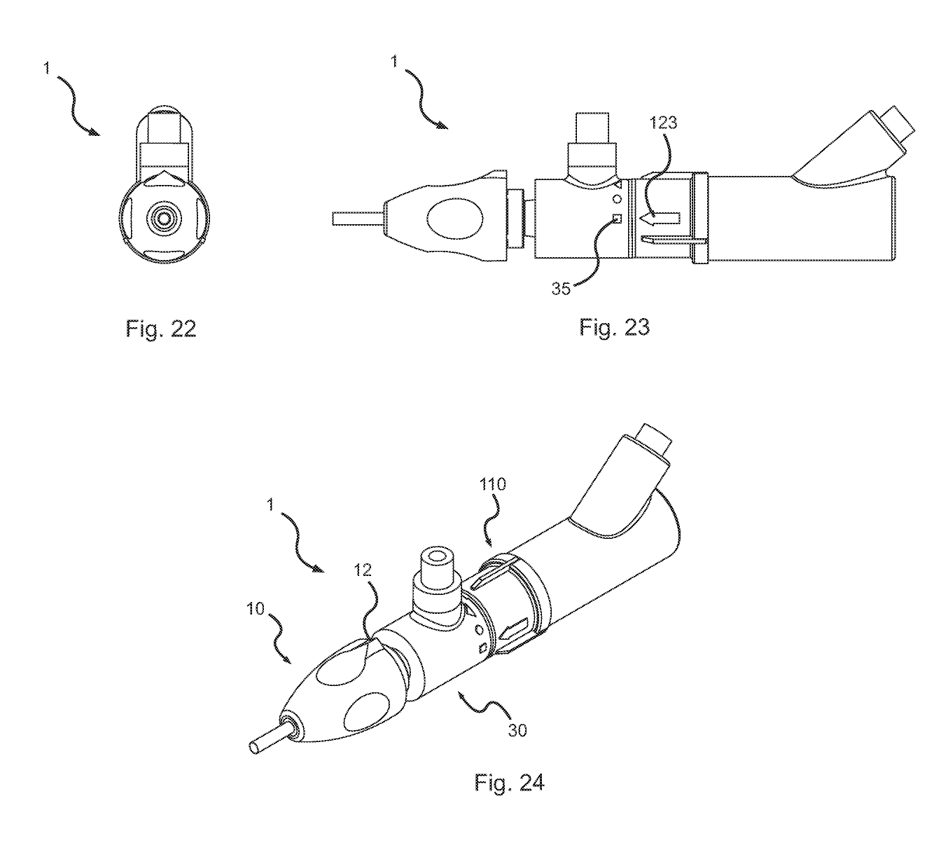

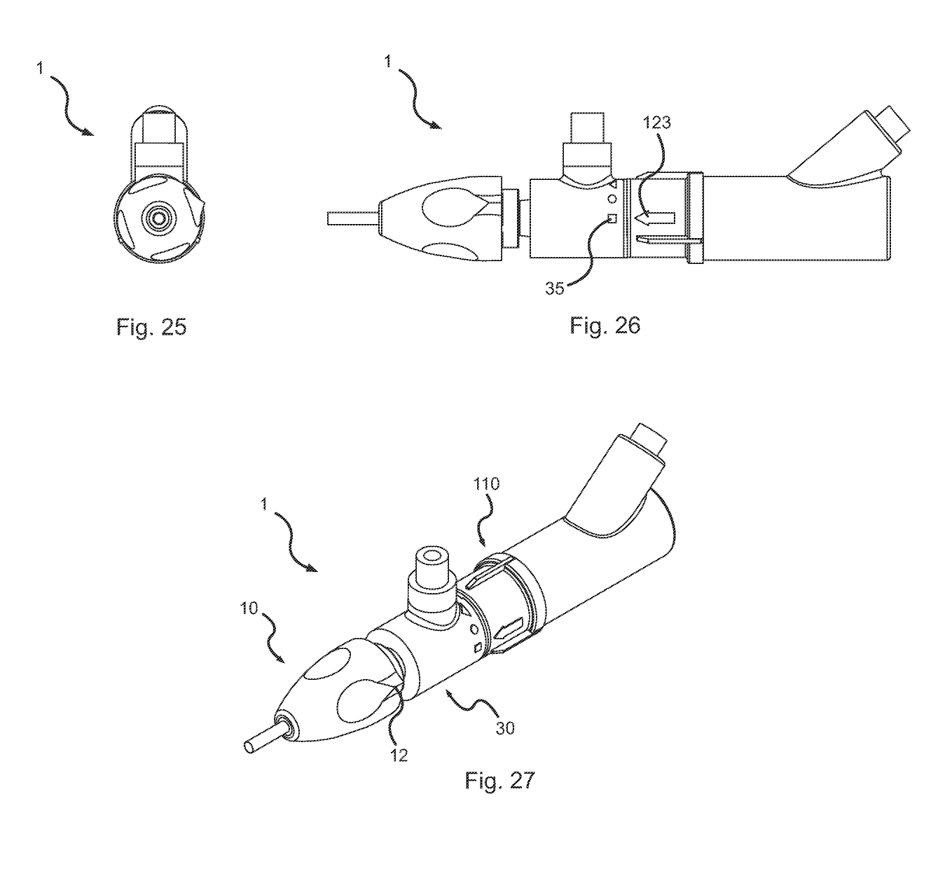

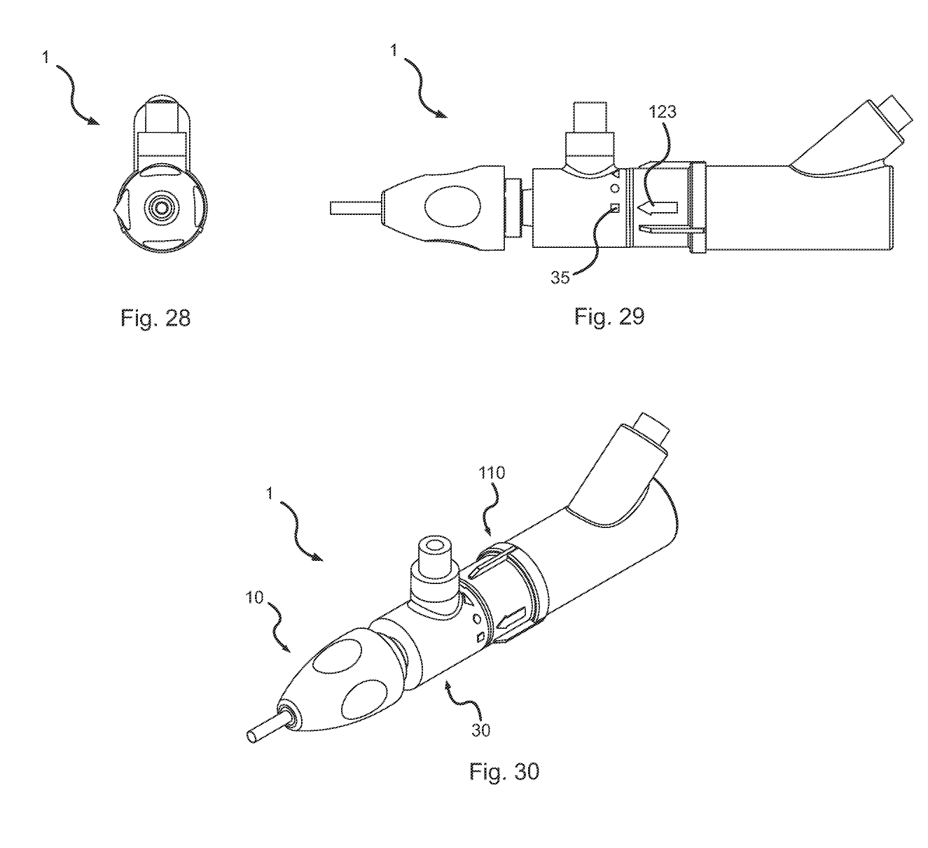

As described previously and as depicted further in FIGS. 22-30, hub 10 and its protruding ridge 12 rotate independent of the rotation of straight ported bobbin half 30 and valve bobbin half 50 around main valve body with port 110. As depicted in FIGS. 22-24 strain relief and distal control member or hub 10 may be kept in an aligned position with reference to protruding ridge 12. Note that module 1 is in a working mode of operation as shown by indexing marks arrow 123 and square 35. In FIGS. 25-27, strain relief and distal control member or hub 10 can be rotated to a right or clockwise rotated position with reference to protruding ridge 12. Note that module 1 has remained in a working mode of operation as shown by indexing marks arrow 123 and square 35. In FIGS. 28-30, strain relief and distal control member or hub 10 can be rotated to a left or counter-clockwise position with reference to protruding ridge 12. Note that module 1 has remained in a working mode of operation as shown by indexing marks arrow 123 and square 35.

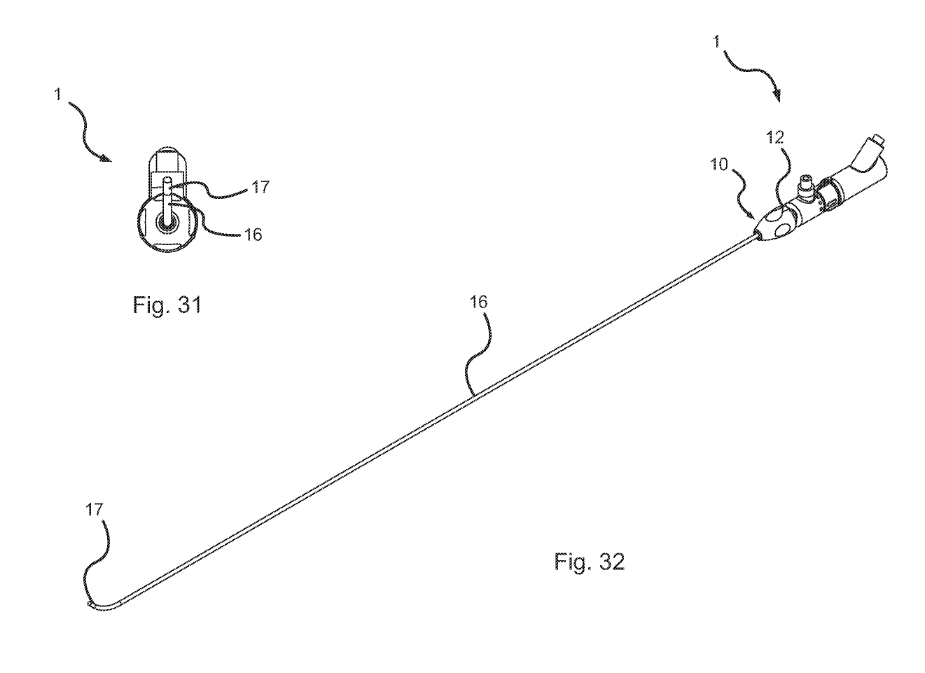

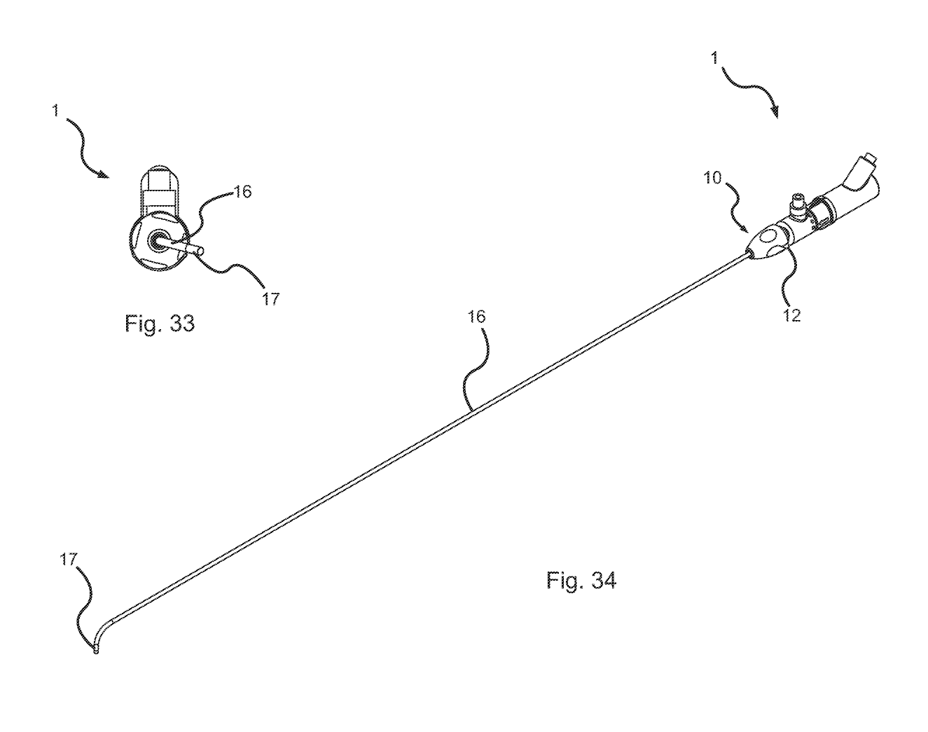

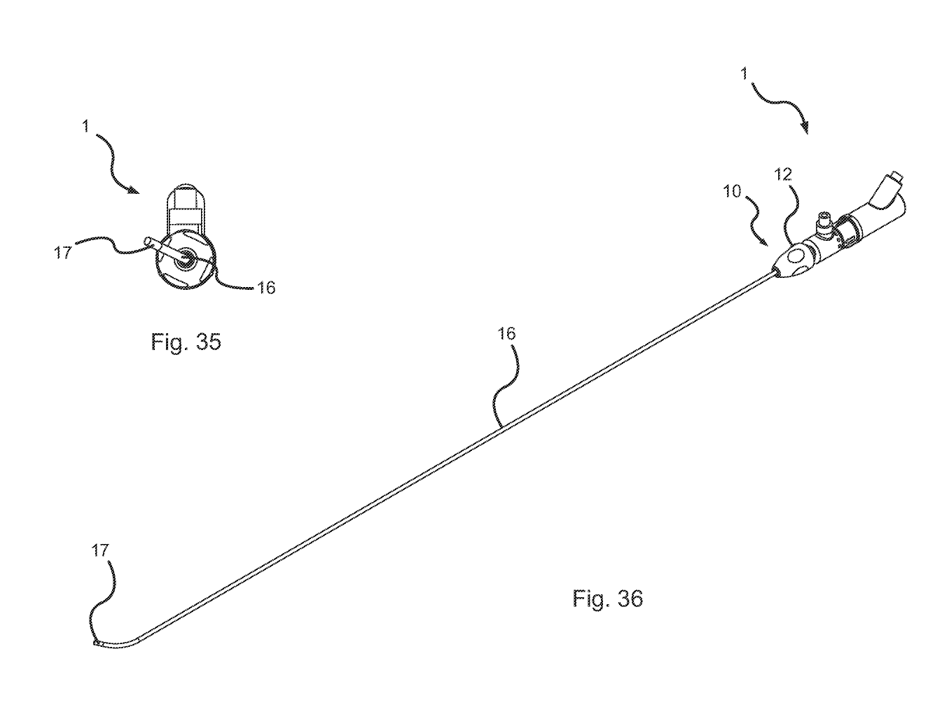

As depicted further in FIGS. 31-36, sheath 16 with its distal curved tip 17 (because it is fixedly attached to hub 10) can rotate independent of the rotation of straight ported bobbin half 30 and valve bobbin half 50 around main valve body with port 110. This can be very useful during many procedures and techniques where a distal tip deflection may be required. Sheath 16 with its distal curved tip 17 (because it is fixedly attached to hub 10) can rotate from 0 through 360 degrees. As seen in FIGS. 31-32, sheath tip 17 may be kept in an aligned position with reference to protruding ridge 12. As seen in FIGS. 33-34, sheath tip 17 can be rotated to a right or clockwise rotated position with reference to protruding ridge 12. in a right or clockwise rotated position. As seen in FIGS. 35-36, sheath tip 17 can be rotated to a right or clockwise rotated position with reference to protruding ridge 12. Note that the orientation of sheath tip 17 and protruding ridge 12 are always the same and ridge 12 therefore indicates by its position a direction of curvature of tip 17. Thus, no matter where sheath tip 17 is located or positioned during a procedure or technique, a user will always know the orientation of tip 17 because it corresponds directly to the orientation of protruding ridge 12 of hub 10 in the user's hand, which the user can tactically feel.

In further describing the operation of laminar valve flow module implementations, and for the exemplary purposes of this disclosure, laminar valve flow module 1 may be used to introduce various cardiovascular devices, such as catheters, into the left side of the heart through the interatrial septum.

Careful consideration should be taken to reduce the potential dangers associated with the transseptal technique such as air emboli or perforation of the aorta or the left atrium. Only those physicians who are specifically trained in the transseptal procedure should attempt to use module 1. Fluoroscopy should be used to confirm the positioning throughout the procedure. Transseptal procedures should be performed in facilities equipped and staffed to perform this procedure. Lab capabilities should include, but are not limited to: Intracardiac pressure monitoring capabilities; Systemic pressure monitoring; Contrast media injection and management of reaction to the contrast media; Pericardiocentesis; Surgical backup; and Anticoagulation therapy and monitoring

The user should maintain monitoring of vital signs throughout the procedure and inspect all components before use. Only Brockenbrough Needles should be used with a stylet that contains an appropriate curve for transseptal procedures. Prior to using module 1 with a patient, the dilator should be preassembled through the sheath 16. Advance the Brockenbrough needle through the dilator to check for excessive resistance as the tip of the needle advances through the curvature of the sheath/dilator assembly. During insertion, caution should be taken not to create excessive bends in the device. This may inhibit the advancement of the needle and may result in inadvertent needle puncture of the sheath/dilator. During insertion, the stylet should always be used in order to facilitate needle passage through the sheath/dilator assembly or skiving of material from the inter surface of the dilator.

To minimize the potential for creating a vacuum in the sheath 16, remove and make catheter exchanges slowly. Always prime the system with saline and ensure all visible air is removed. Once the sheath 16 is inserted in the vasculature and the dilator is removed, aspirate until steady blood returns to the outflow port 32. All fluid infusions should be made through the “In” port 92.

Aspirate when removing the catheter or dilator in order to minimize embolic risk, provide continuous infusion of heparinized saline through the “In” port 92 during the procedure to minimize embolic risks. Do not remove the dilator or catheter rapidly. Damage to the valve may occur. If resistance is met when advancing or withdrawing guidewire or introducer, determine the cause and take corrective action before continuing with the procedure.

Indwelling intracardiac introducer sheaths should always be supported with a catheter or an obturator. Do not manipulate the sheath in the heart without a catheter or obturator.

With the foregoing considerations in mind, the following is the suggested transseptal procedure using laminar valve flow module 1. There are eight general steps in the transseptal procedure: Prepare and assemble equipment; Advance sheath/dilator assembly over a guide wire into the Superior Vena Cava; remove the guide wire; Position selected Brockenbrough Needle inside the assembly; Drag the assembly and engage the fossa ovalis; Puncture the fossa ovalis with the Brockenbrough Needle; Advance sheath/dilator assembly over the fixed dilator and needle into left atrium; and Remove needle and replace with catheter.

First, preparing module 1 requires the following items: an appropriate Brockenbrough Needle with stainless steel stylet; a 0.032 150-180 cm guidewire with 3 mm “J” tip; 10 CC syringes for aspirating and flushing; a collection bag for aspirated blood, saline and bubbles; sterile heparinized saline; and two- or three-way Stop-cocks.

Module 1 is then removed from its sterile packaging using the aseptic technique. Take care not to bend sheath 16. Next, a 10 CC syringe is filled with heparinized saline and connected to “In” port 92. Then, a collection bag with stop-cock is connected to the “Out” port 32. With the stop-cock in the “Open” position, and valve body 110 turned to the prime position (arrow 123 pointing to the triangle 33), prime chambers 150 and 160 with heparinized saline using the syringe until all air has been evacuated and saline flows into the collection bag.

Next, with sheath 16 elevated, turn main valve body 110 to the working position (arrow 132 is lined up with square 35) such that flow is cut off into collection bag and continue flushing with syringe until heparinized saline flows out of sheath 16. This will flush unwanted air out of distal chamber 170 and sheath 16. The, remove the syringe and connect a heparinized saline bag to “In” port 92. Saline should be present in all of the chambers, holes, and channels of module 1. No air should be present and saline flush should flow freely. Do not remove the saline lines. When module 1 is completely primed, air free saline flow is controlled by adjusting the supply.

Next, flush the dilator with heparinized saline. Insert the primed dilator in module 1 and flush. With the Brockenbrough valve open, remove the stylet from the Brockenbrough needle and flush with heparinized saline. Reinsert the stylet into the primed Brockenbrough and lock the stylet into the hub. Then, insert the primed Brockenbrough needle and stylet into the dilator and then into module 1.

Next, withdraw the tip of the Brockenbrough needle into the sheath 16/dilator. The end of the dilator has a radiopaque marker. Measure the distance from the Brockenbrough pointer flange and the dilator hub 10. Record this measurement for use during the procedure. It is critical during the procedure to maintain the distance between the pointer flange and dilator hub 10. This insures that the needle assembly does not extend beyond the dilator tip until it is deployed or transseptal crossing.

Then, remove the Brockenbrough needle from the dilator. Remove the stylet from the Brockenbrough needle and flush the needle again. Reinsert and lock the stylet. Flush the dilator again And finally, check for air in system.

Second, to advance the sheath 16/dilator assembly into the superior vena cava, obtain femoral access with a venous sheath using the Seldinger Technique (right femoral preferred). Introduce a 0.023′ guidewire, 150-180 cm, 3 mm “J” tip guidewire into the superior vena cava. Insert the sheath 16 and dilator assembly over the guidewire and advance the assembly into the superior vena cava (SVC). Once the dilator is in the SVC, confirm the point is pointed medially.

Third, to position the selected Brockenbrough needle and stylet assembly inside the sheath/dilator assembly, remove the guidewire from the dilator. Aspirate blood from the dilator and withdraw the dilator slowly to avoid entering the blood stream. Flush the Brockenbrough needle with clean heparinized saline, insuring that no air remains in the system to insure that no air can enter the bloodstream. Separate the dilator and sheath by withdrawing the dilator (while aspirating) and while holding the sheath 16 and module 1 in position. Withdraw the dilator a distance to accommodate the curve. Insure the stylet is locked onto the hub of the Brockenbrough needle. Then insert the Needle into the dilator, letting the needle rotate freely as it advances.

After the Needle curve advances beyond the sheath 16, reposition the sheath 16 over the dilator while maintaining position over the SVC but inside the sheath 16. Advance the needle and stylet until the pointer flange is at the predetermined distance from the dilator hub. Remove the stylet and set aside (do not discard). Attach a syringe to the “Out” Port 32 and rotate main body valve 110 to the Aspirate position (arrow 132 is pointing to the circle 34). Aspirate until blood return is observed. Discard the syringe. Finally, insure no air is trapped in module 1 and rotate main body valve 110 to working position (arrow 132 pointing to square 35).

Fifth, to engage the fossa ovalis, visualize and identify anatomic landmarks. Set the fluoro unit at the appropriate angle, parallel to the mitral valve and orthogonal to the plane of the septum. This will typical be LAO, approximately 30 to 40 degrees. Placing catheters in the coronary sinus (CS) and HIS position can facilitate the identification of anatomic landmarks. In the appropriate LAO view the HIS catheter will appear in profile, the CS catheter will be seen in profile. In the appropriate RAO view the HIS. As an option, a pigtail catheter can be placed in the non-coronary cusp of the aortic root can facilitate the identification of anatomic landmarks. Observe the pressure waveform being recorded through the Brockenbrough Needle. Adjust the needle pointer so that the needle is perpendicular to the fossa ovalis (typically between 3:00 and 5:00 o'clock, as viewed from the foot of the patient). Also confirm that the needle tip is inside the dilator by fluoroscopy and by previous measurement. After confirming the tip of the needle is within the dilator, drag the assembly slowly. Prevent any movements of the assembly parts relative to each other. It is critical to maintain the previous orientation of the needle points. Observe the tip of the dilator during the drag medial (or rightward), indicating the tip has engaged the fossa ovalis. If pressure is being monitored, note that the pressure through the needle will not be accurate at this point, since the tip is against fossa ovalis. If the fossa ovalis is “probe” patent, the dilator tip will move into the left atrium with ease.

Sixth, to puncture the fossa ovalis with the brockenbrough needle, confirm the correct location of the needle in the fossa ovalis before advancing the needle. Once the correct location is confirmed, advance the needle across the interatrian septum. Under pressure monitoring, entry into the left atrium is confirmed when the pressure tracing shows a left atrial pressure tracing. Left atrial access can be confirmed with contrast injection. If there is resistance to needle advancement, re-evaluate the anatomic landmarks. If pericardial or aortic entry occurs, do not advance the dilator over the needle. If the needle has penetrated the pericardium or aorta, it must be withdrawn. Monitor vital signs closely.

Seventh, to advance the sheath/dilator assembly, the sheath/dilator assembly needs to be advanced over the needle while maintaining a fixed needle position. Then, withdraw the needle into the dilator until it is inside the radiopaque tip. Maintain the position of the needle and dilator across the septum. With the dilator in affixed location, advance the sheath over the dilator.

Eighth, in withdrawing the Brockenbrough needle and the dilator, exercise caution since there is risk of air embolism when withdrawing objects from the sheath. Take precautions to prevent air filtration. Disconnect any attachments to the needle hub while maintaining an air seal. Withdraw the Brockenbrough needle from the dilator. Immediately attach a syringe to the “Out” port 32 and aspirate. The blood should be arterial blood. Once the dilator is removed, aspirate through the “Out” Port 32. The sheath 16 is now ready to use.

1. A laminar valve flow module configured for preventing gas from passing through a medical device into a patient's cardiovascular system, the laminar valve flow module comprising:
a proximal chamber, a purge chamber, and a sheath chamber in fluid communication with each other and between an inlet opening and an exit opening, the sheath chamber in fluid communication with the exit opening; and
a guide chamber located within the proximal chamber and in fluid communication with the inlet opening, the proximal chamber, and the sheath chamber.

2. The laminar valve flow module of claim 1 further comprising a distal rotating connector in fluid communication with and rotatably coupled to the exit opening and configured to couple with a sheath.

3. The laminar valve flow module of claim 2 wherein the distal rotating connector rotates within a 360 degree range.

4. The laminar valve flow module of claim 2 wherein the distal rotating connector comprises a ridge for indicating a direction of curvature of a medical device.

5. The laminar valve flow module of claim 2 further comprising a rotating valve assembly that rotates freely and independent from the distal rotating connector.

6. The laminar valve flow module of claim 1 further comprising a rotating valve assembly.

7. The laminar valve flow module of claim 1 wherein the proximal chamber is a pliable chamber.

8. The laminar valve flow module of claim 1 wherein the guide chamber has a tapered distal portion.

9. A laminar valve flow module configured for preventing substantial infusion of air into the proximal end of a sheath comprising:
means for collecting air within an inner catheter guide chamber; means for collecting air within a proximal outer chamber; means for permitting the air to move from the inner catheter guide chamber to the outer proximal chamber; means for inserting a catheter through the inner catheter guide chamber; means for preventing substantial air from entering the catheter guide chamber from the proximal end of the inner catheter guide chamber; means for preventing substantial air from leaving the inner catheter guide chamber at its distal end while still permitting passage of a catheter there through; means for infusion of fluid into the outer proximal chamber; and means for removal of gas from the outer proximal chamber.

10. The laminar valve flow module of claim 9 further comprising a means for rotating a distal connector within a 360 degree range.

11. The laminar valve flow module of claim 9 further comprising a means for indicating a direction of curvature of a medical device.

12. A method of preventing substantial infusion of air into the proximal end of a sheath comprising:
affixing a laminar valve flow module to the proximal end of a first catheter;
affixing a source of sterile liquid to the laminar valve flow module;
affixing a gas withdrawal system to the laminar valve flow module;
inserting a secondary catheter through the laminar valve flow module into the first catheter without air entering or escaping the laminar valve flow module; and
removing gas bubbles that collect in the laminar valve flow module.
